(19) United States
(12) Patent Application Publication
(10) Pub. No.: US 2012/0232290 A1
(43) Pub. Date: Sep. 13, 2012

(54) REACTIVITY OF HYDROXYMETHYLGLUTARYL COENZYME A (HMG-COA) REDUCTASE INHIBITORS CONTAINING CONJUGATED DIENES WITH PHENOLIC ANTIOXIDANTS IN THE SOLID-STATE

(76) Inventor: Russell F. Lang, Pembroke Pines, FL (US)

(21) Appl. No.: 13/437,330

(22) Filed: Apr. 2, 2012

Related U.S. Application Data
(63) Continuation-in-part of application No. 12/139,222, filed on Jun. 13, 2008, now abandoned.
(60) Provisional application No. 60/943,885, filed on Jun. 14, 2007.

(51) Int. Cl. C07D 309/30

(52) U.S. Cl. 549/292

(57) ABSTRACT

The object of this invention was to probe the reactivity of lovastatin, simvastatin, pravastatin, and mevastatin in the solid-state without radical initiators in order to determine the antioxidant that provided the best stability for the statin. Phenolic antioxidants were evaluated.

This application claims priority from Provisional U.S. Patent Application Ser. No. 60/943,885 filed Jun. 14, 2007, which is incorporated by reference in its entirety.

### BACKGROUND OF THE INVENTION

Oxidative degradation is a common mechanism for the degradation of drugs. This degradation limits the shelf life of pharmaceutical products and may produce unknown degradates or mass balance deficiencies. Initiation of autoxidation reactions are generally attributed to several different processes which include thermal or photochemical cleavage of a R—H bond, reaction with metal ions, and hydrogen atom abstraction by a free radical.

Lovastatin, simvastatin, pravastatin, and mevastatin are hydroxymethylglutaryl coenzyme A (HMG-CoA) reductase inhibitors, also termed statins, and contain a heteroannular diene ring system that is potentially susceptible to oxidation. Antioxidants have found use in pharmaceutical formulations to reduce oxidative drug degradation. The antioxidants produce a radical chain termination in the free radical reaction. Antioxidants in the traditional sense are substances that interrupt the propagation step of radical reactions. Oxidation of lovastatin in the solid-state was shown to be inhibited by natural antioxidants including caffeic acid, rutin, quercetin, gallic acid and ascorbic acid.

Solid-state reactions in formulations is a challenge in pharmaceutical research and development. Oxidation and other reactions are common degradation pathways encountered in the solid-state. Antioxidants are commonly used to provide stability to a formulation however, as this work showed, careful selection of the antioxidant or combination of antioxidants is essential since the antioxidant-effect may be specific to the active drug. Additionally, minor changes in formulation components could potentially alter the reactivity of the drug or drug/antioxidant system. Prooxidant effects are difficult to predict and must be considered during formulation development. Evaluation of the reactivity of the drug and candidate antioxidant in the absence of excipients provides basic information related to potential reactivity problems.

### SUMMARY OF INVENTION

The object of this invention was to probe the reactivity of lovastatin, simvastatin, pravastatin, and mevastatin in the solid-state without radical initiators in order to determine the antioxidant that provided the best stability for the statin. This approach was developed to examine how these statins are affected by antioxidants without the influences of other excipients typically used in solid dosage forms, since a number of excipients are known to contain hydroperoxyl impurities. The structures of the statin compounds and antioxidants studied are shown in FIGS. 1 and 2 respectively.

In particular, one embodiment of the present invention includes the use of butylated hydroxy anisole (BHA) to reduce oxidative degradation of simvastatin.

Another embodiment of the present invention includes the use of propyl gallate to reduce oxidative degradation of simvastatin.

Another embodiment of the present invention includes the use of α-tocopherol to reduce oxidative degradation of simvastatin.

In particular, one embodiment of the present invention includes the use of butylated hydroxy anisole (BHA) to reduce oxidative degradation of lovastatin.

Another embodiment of the present invention includes the use of propyl gallate to reduce oxidative degradation of lovastatin.

Another embodiment of the present invention includes the use of α-tocopherol to reduce oxidative degradation of lovastatin.

In particular, one embodiment of the present invention includes the use of butylated hydroxy anisole (BHA) to reduce oxidative degradation of pravastatin.

Another embodiment of the present invention includes the use of propyl gallate to reduce oxidative degradation of pravastatin.

Another embodiment of the present invention includes the use of α-tocopherol to reduce oxidative degradation of pravastatin.

In particular, one embodiment of the present invention includes the use of butylated hydroxy anisole (BHA) to reduce oxidative degradation of mevastatin.

Another embodiment of the present invention includes the use of propyl gallate to reduce oxidative degradation of mevastatin.

Another embodiment of the present invention includes the use of α-tocopherol to reduce oxidative degradation of mevastatin.

### BRIEF DESCRIPTION OF THE DRAWINGS

FIG. 1 shows structures of the statin compounds.

FIG. 2 shows the structures of the antioxidants.

FIGS. 3A and 3B show reversed-phase chromatograms of the solid-state reactions of simvastatin with BHA and simvastatin with α-tocopherol respectively.

FIG. 4 shows the negative ion ESI mass spectra.

FIG. 5 shows the structures of the identified degradates.

FIG. 6 displays superimposed chromatograms of the two samples: simvastatin/BHA and simvastatin experimental formulation.

FIG. 7 shows a comparison of the ultraviolet spectrum for 3(S)-hydroperoxysimvastatin from the two samples: simvastatin/BHA and simvastatin experimental formulation.

FIG. 8 shows the negative and positive ESI spectra of the for the two samples: simvastatin/BHA and simvastatin experimental formulation.

FIG. 9 shows the reaction profiles for the four statins with the antioxidants.

FIG. 10A shows the plot of total hydroperoxyl degradates in the solid phase at 50° C. versus time. 10B shows the plot of 6-hydroxysimvastatin in the solid phase at 50° C. versus time.

FIG. 11 shows the decrease of simvastatin in reactions of simvastatin with BHA at different molar ratios.

FIG. 12 shows the formation of hydroxyl and hydroperoxyl degradates of simvastatin and BHA.

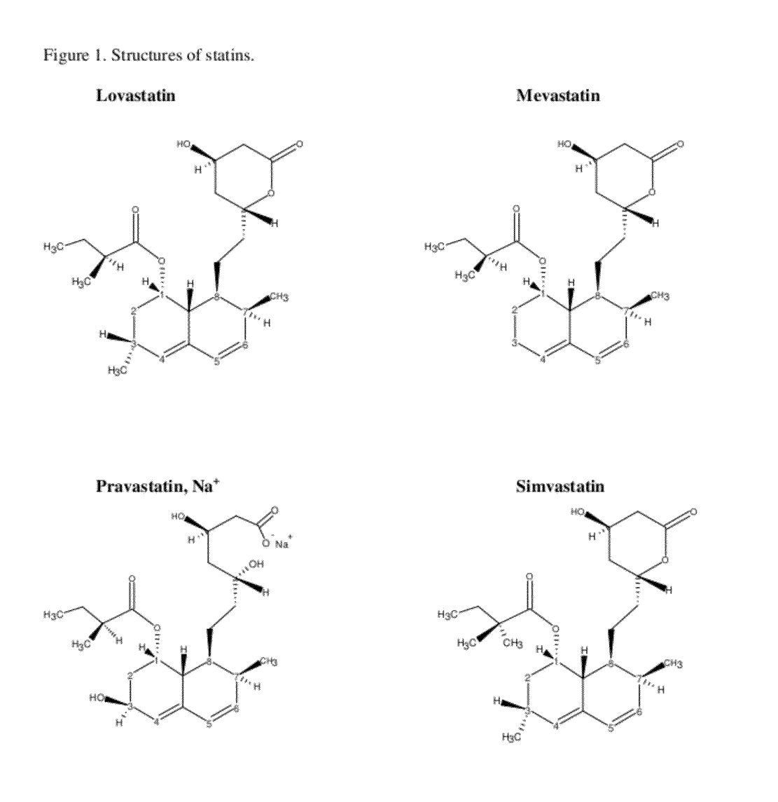

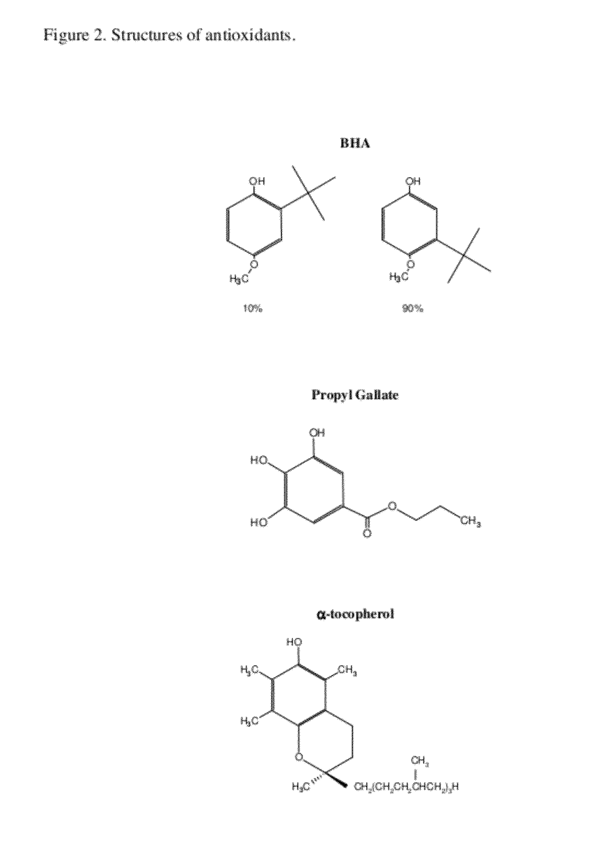

### DETAILED DESCRIPTION

### Example 1

Simvastatin, lovastatin, sodium pravastatin and mevastatin were obtained from Betachem Inc, Upper Saddle River, N.J. α-tocopherol (98%) was obtained from Sigma-Aldrich, Milwaukee, Wis.; Propyl gallate (100.4%) from Spectrum Chemicals and Laboratory Products, New Brunswick, N.J.; BHA (100.2%) from Penta Manufacturing Co, Livingston, N.J.

Reactions of Statins with Antioxidants

Typical preparations entailed adding about 0.1 mmoles of statin with 0.05 mmoles of antioxidant to a 250 mL beaker then dissolving the material with 20 mL of suitable solvent. Solvents used varied due to the solubility of the statin. Reactions with simvastatin were performed in acetonitrile, lovastatin (1:1 acetonitrile:methanol), mevastatin (1:1 dichloromethane:methanol) and pravastatin (80:20 acetonitrile:water). The beaker was placed in a dark oven maintained at 50° C. overnight. The beaker was removed from the oven, allowed to cool, then 20 mL of solvent were added and the beaker swirled to redissolve the solid. The beaker was returned to the oven and heated an additional 24 h at 50° C. This dissolution and heating process was repeated daily for the duration of the specific experiment. Reactions of simvastatin with different ratios of BHA were performed in acetonitrile and processed as described above.

### Reversed-Phase HPLC Analytical Method

Reversed-phase HPLC analyses were performed using an Agilent 1100 HPLC with an Agilent diode array detector acquiring data in the range of 205-400 nm. This LC/MS-compatible method utilized a Discovery RP Amide C₁₆ 250 mm×4.6 mm, 5 μm column (Supelco Inc, Bellefonte Pa.). Mobile phase A was aqueous 0.1% (v/v) formic acid and mobile phase B was acetonitrile containing 0.1% (v/v) formic acid. Mobile phase flow rate was 1 mL/min and the column was maintained at 30° C. The elution sequence was isocratic 60% A:40% B for 0.5 min; a gradient of 60% A:40% B to 50% A:50% B over 1.5 min then hold for 28 min; 50% A:50% B to 20% A:80% B over 1.0 min then hold for 3 min; 20% A:80% B to 100% B over 1.0 min then hold for 4 min. LC/MS analyses were performed using a Waters Acquity UPLC with PDA and a Micro Quattro MS with infusion pump with the LC method described above.

### Isolation and Purification of Reaction Products

The solid from the statin/antioxidant reactions was typically dissolved in dichloromethane. Aliquots of this solution were injected into a Waters Preparative LC system. This system consisted of a 2525 Binary Gradient Module, 2996 PDA, and 2767 Sample Manager and was used for all preparative LC work. Isolation of the reaction products from starting materials was performed with a Phenomenex LUNA Silica, 5 μm, 21.2 mm×250 mm column using a 5.0 mL injection volume and an isocratic elution of 2% methanol/dichloromethane at a flow rate of 42 mL/min. Detection was total absorbance from 200-300 nm. Collected fractions were concentrated using a roto-vap. The individual compounds were isolated using the Phenomenex LUNA Silica column with a gradient method of 8% ethanol/92% isooctane to 10% ethanol/92% isooctane over 10 min with a 5 min final hold. Fractions were analyzed for purity using a Shimadzu LC20AB HPLC with Dual Wavelength UV/VIS Detector with mobile phases of isooctane (A) and ethanol (B), and a Phenomenex LUNA Silica, 250 mm×4.6 mm, 5 μm column. The gradient was 90:10 (A:B) to 65:35 over 10 min. The column temperature was ambient, with a mobile phase flow rate of 2 mL/min, an injection volume of 25 μL and detection at 238 nm.

### NMR Analyses

NMR analyses were performed using a Bruker Spectrospin, 400 Ultrashield, Model Advance 400 NMR Spectrometer with a 5 mm QNP probe or a 600 MHz Ultrashield Model Advance NMR spectrometer with a TBI broadband probe. ¹H, ¹³C, DEPT 135, 45, 90, COSY, HMBC, HMQC and HETCOR experiments were used for the characterization of the isolates. NOE differences and NOESY were used to elucidate stereochemistry. Results were analyzed using FELIX (Accelrys, 10188 Telesis Court, San Diego, Calif. 92121) data analysis software. Chemical shifts (6) are expressed in ppm downfield from tetramethylsilane (internal standard). Isolates were dissolved in CD3CN for all analyses.

Mass Spectral Analysis of Isolates. Molecular Weight Determinations

The isolates were analyzed by positive and negative ESI mass spectrometry to determine molecular weight. The isolates, dissolved in CD₃CN were infused into the mass spectrometer at a rate of 10 μL/min. Co-infused with the sample was a 1:1 solution of 0.1% aqueous formic acid:0.1% formic acid in acetonitrile at a rate of 0.2 mL/min. The formic acid enhanced formation of formate adducts in negative ESI mode for most analytes which facilitated spectral interpretations.

### Comparison of LC/MS and LC/UV Spectra of Simvastatin/BHA Reaction Product to Experimental Simvastatin Formulation

Ultraviolet and mass spectral characteristics of chromatographic peaks, using the reversed-phase analytical method, from a simvastatin/BHA reaction sample were compared to those from an extract of an experimental simvastatin formulation also containing BHA. The extract of an experimental drug formulation was prepared by dissolving 10 tablets in 100 mL acetonitrile. After stirring for 6 h, a 10 mL aliquot was withdrawn and filtered through a 0.45 μm syringe filter. The extract volume was reduced to 1 mL at ambient temperature under a stream of helium. Ultraviolet, positive and negative ESI spectra were obtained for both samples. The reversed-phase HPLC Analytical Method was used with the PDA detector scanning the range of 200-400 nm. MS parameters used were Source Temperature: 80° C.; Desolvation Temperature: 250° C.; Cone Gas Flow: 50 l/h; Desolvation Gas Flow: 800 l/h; Capillary: 3.84 kV (ESI+), 2.44 kV (ESI−); Cone: 14.00 V (ESI+), 28.00 V (ESI−).

### RESULTS

### Identification of Reaction Products

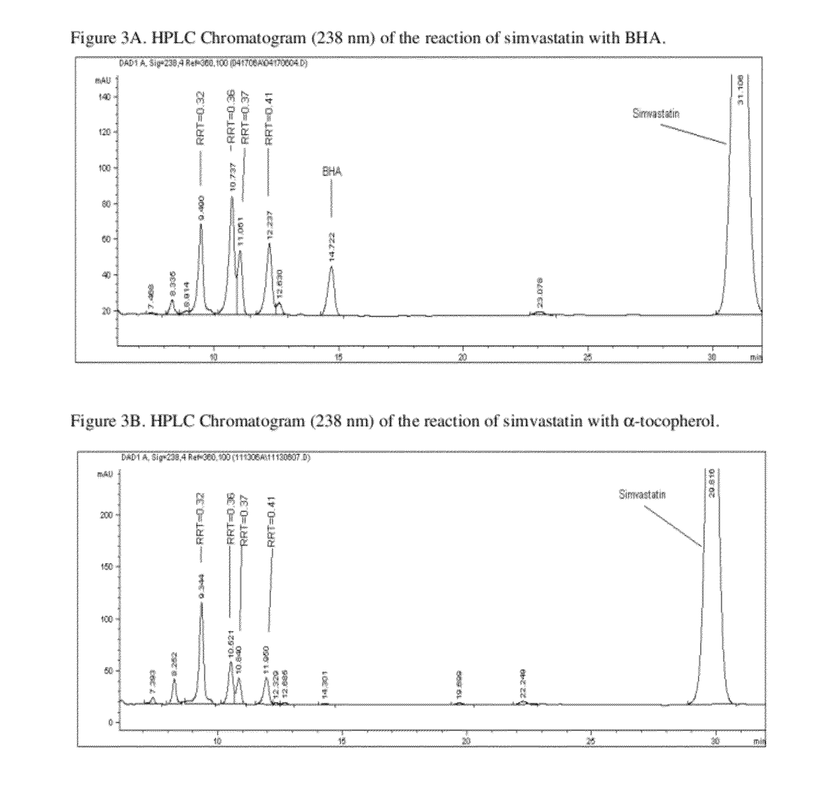

Of the combinations of statins and antioxidants studied, the reaction of simvastatin with BHA and α-tocopherol produced the highest concentration of product peaks. FIGS. 3A and 3B are reversed-phase chromatograms of the solid-state reactions of simvastatin with BHA and simvastatin with α-tocopherol respectively at 50° C. after five days. The four major peaks produced from this reaction are distinguished by relative retention time (RRT). Peaks with identical retention times were also observed in some experimental simvastatin formulations.

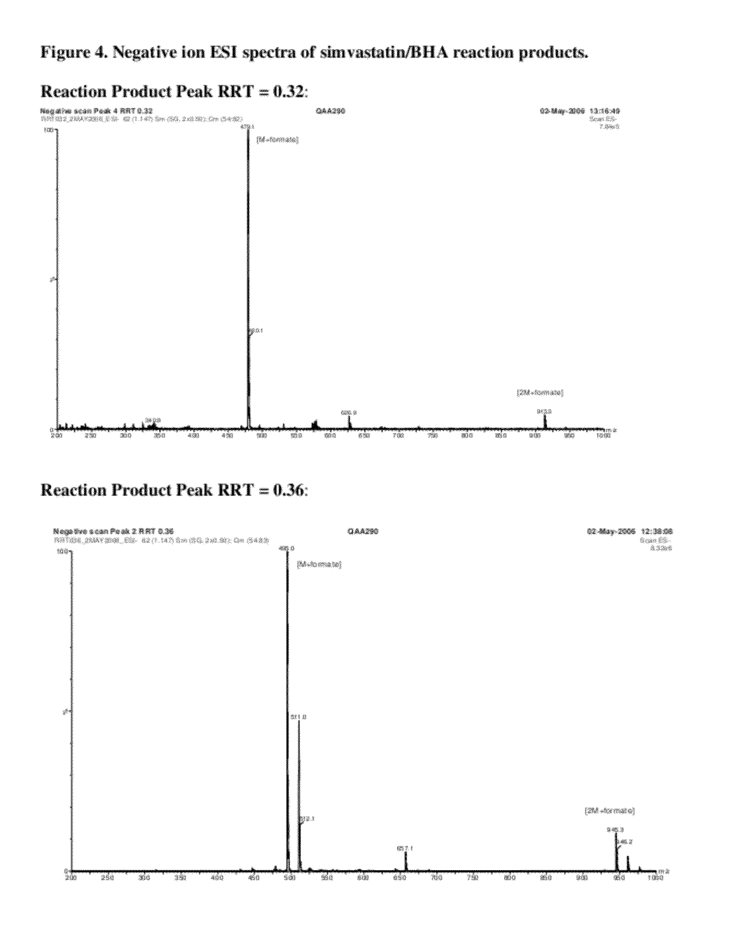

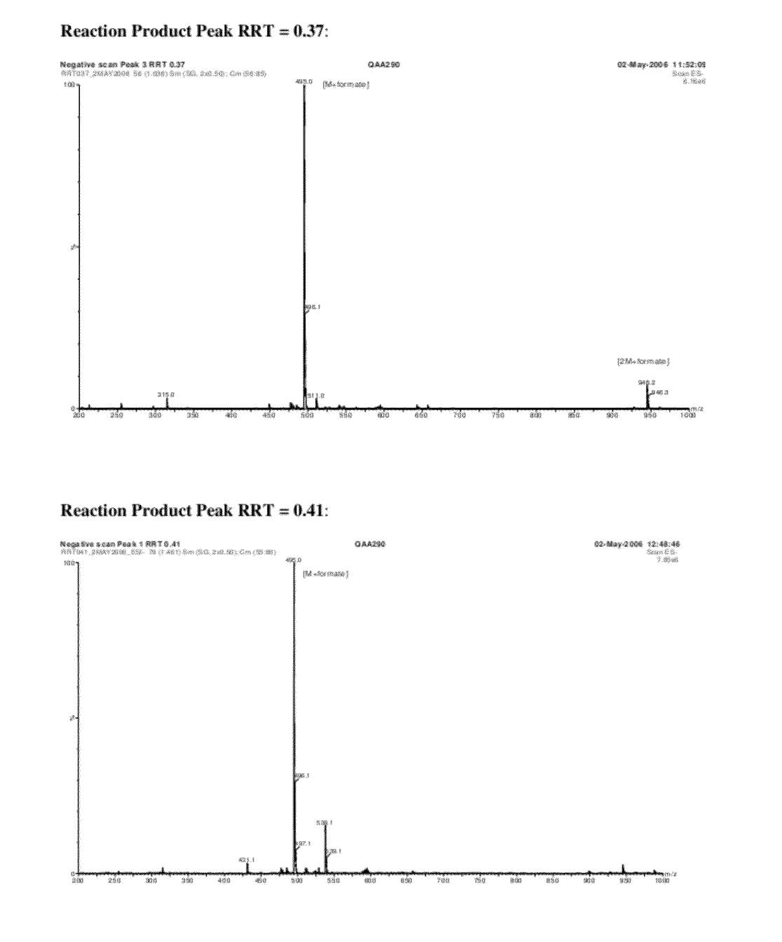

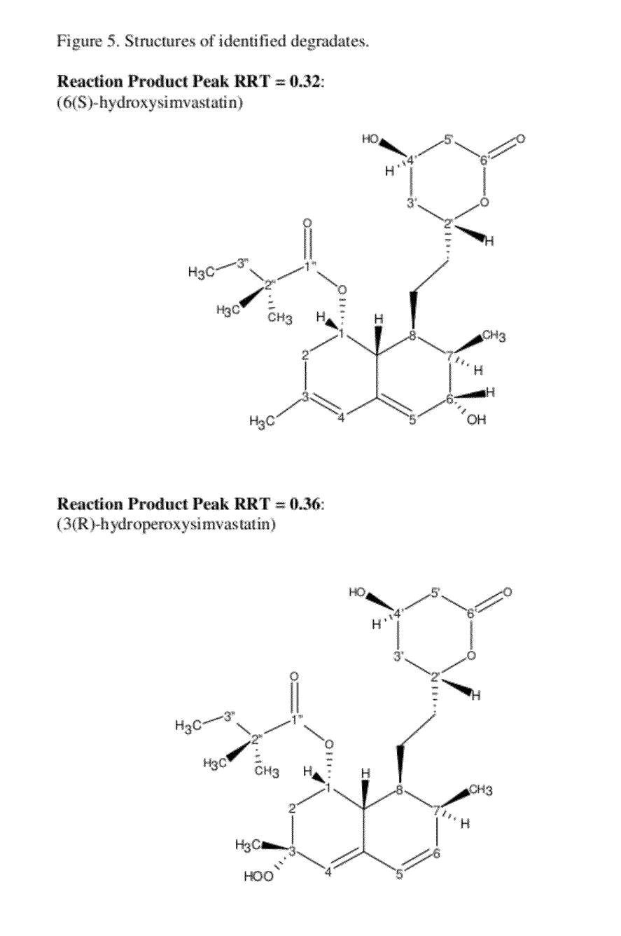

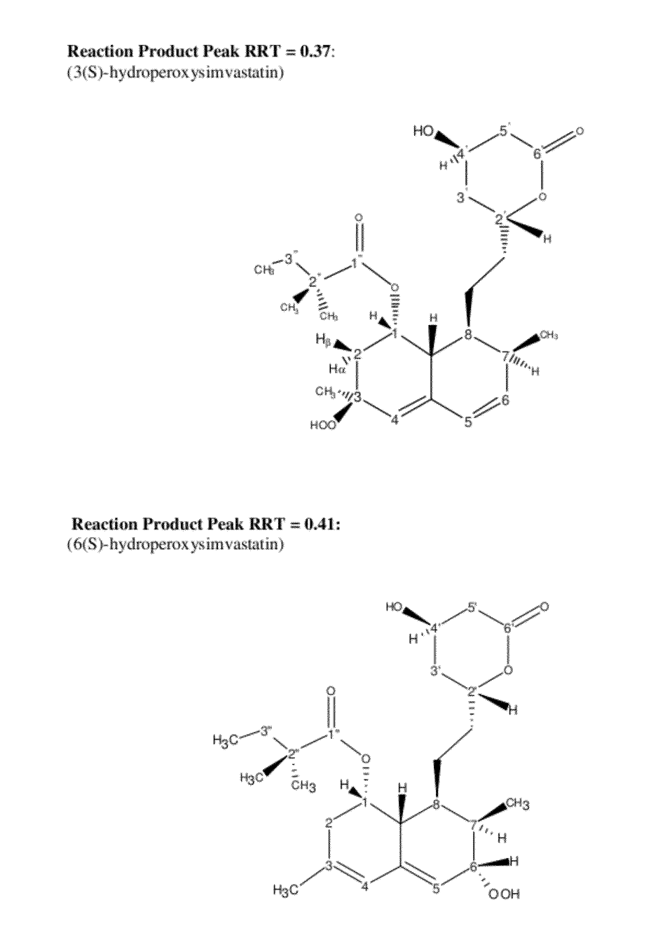

Negative ion ESI mass spectra for the four peaks are shown in FIG. 4. Co-infusion of formic acid mobile phase with samples isolated chromatographically from the reaction mixture produced prominent [M+formate]⁻ ions. These analyses indicated that the molecular weight for unknown RRT 0.32 was 434 Da and the molecular weights for peaks RRT 0.36, RRT 0.37, and RRT 0.41 were 450 Da. Positive ESI spectra of the isolates showed prominent [M+H]⁺ or [M+NH₄]⁺ ions supporting the molecular weight estimates from the negative ion spectra. These molecular weight results suggested the addition of one oxygen atom to simvastatin (MW=418 g/mol) for the RRT 0.32 product and the addition of two oxygen atoms to simvastatin for the RRT 0.36, RRT 0.37, and RRT 0.41 products. Characterization of the isolates by NMR established that these compounds were mono-hydroxy and mono-hydroperoxy derivatives of simvastatin. The 0.32 RRT degradate was determined to be 6(S)-hydroxysimvastatin. The degradates at RRT 0.36, 0.37, and 0.41 were the 3(R)-hydroperoxyl, 3(S)-hydroperoxyl and 6(S)-hydroperoxyl derivatives of simvastatin respectively. NMR results for 6(S)-hydroxy and 6(S)-hydroperoxy compounds were in agreement with previous reports. FIG. 5 shows the structures of these compounds. The hydoperoxide proton exhibited a characteristic downfield singlet in the ¹H NMR spectrum. For example, the spectrum for 6(S)-hydroperoxysimvastatin displayed the singlet at 9.64 ppm. Upon addition of a drop of deuterium oxide to the NMR sample tube, the signal disappeared due to proton exchange. Additionally, 6(S)-hydroperoxysimvastatin was reduced to 6(S)-hydroxysimvastatin when reacted with NaBH₄. This reduction of the hydroperoxide to the alcohol proceeded with retention of the stereochemistry. These hydropeoxide compounds were relatively unstable in both solid and solution phases. 3(R)-hydroperoxysimvastatin and 3(S)-hydroperoxysimvastatin slowly decomposed to numerous products over a few days even when stored under freezer conditions.

### NMR Data

6(S)-hydroxysimvastatin: 400 MHz ¹H NMR (CD₃CN): δ (ppm) 5.88 [b, 1H, C(4) H], 5.50 [d, 1H, J=4.2 Hz, C(5) H], 5.30 [m, 1H, C(1) H], 4.55 [m, 1H, C(2′) H], 4.20 [m, 1H, C(4′) H], 3.83 [b, 1H, C(6) H], 3.30 [b, 1H, C(4′) OH], 1.73 [s, 3H, C(3) CH₃], 1.06 [s, 3H, C(2″) CH₃], 1.03 [s, 3H, C(2″) CH₃], 1.03 [d, 3H, J=9.96 Hz, C(7) CH₃]. 100 MHz ¹³C NMR: δ (ppm) 178.08 [C(1″)], 171.29 [C(6′)], 137.54 [C(3)], 134.39 [C(4a)], 125.14 [C(5)], 124.09 [C(4)], 76.90 [C(2′)], 70.38 [C(6)], 68.44 [C(1)], 63.03 [C(4′)], 43.55 [C(2″)], 39.94 [C(8a)], 39.24 [C(5′)], 36.70 [C(3′)], 36.61 [C(7)], 36.47 [C(2)], 33.91 [C(10)], 33.83 [C(9)], 32.12 [C(8)], 25.29 [(C(2″-CH₃)], 25.03 [C(3″)], 24.69 [(C(2″-CH₃)], 23.44 [C(3-CH₃)], 11.01 [C(7-CH₃)], 9.66 [C(4″)]. DEPT 135 revealed that C(5′), C(3′), C(2), C(3″), C(9) and C(10) are all methylene groups (CH₂). DEPT 90 indicated that C(5), C(4), C(2′), C(6), C(1), C(4′), C(8a), C(7) and C(8) are all methine groups (CH). 600 MHz COSY indicated the following pairs of ¹H resonances are coupled to each other in the decalin ring: 5.88 [C(4) H] and 5.50 [C(5) H], 5.88 [C(4) H] and 2.44 [C(2) H₂], 5.88 [C(4) H] and 1.73 [C(3) CH₃], 3.83 [C(6) H] and 5.50 [C(5) H], 3.83 [C(6) H] and 1.89 [C(7) H], 2.06 [C(8a) H] and 5.50 [C(5) H], 2.06 [C(8a) H] and 5.30 [C(1) H], 2.06 [C(8a) H] and 1.84 [C(8) H], 5.30 [C(1) H] and 2.44 [C(2) H₂]. HETCOR and HMQC provided the expected correlations of the decalin ring that support the structural assignment of this simvastatin degradant.

6(S)-hydroperoxysimvastatin: 400 MHz ¹H NMR (CD₃CN): δ (ppm) 9.64 [s, 1H, C(6) OOH], 5.90 [b, 1H, C(4) H], 5.38 [d, 1H, J=5.0 Hz, C(5) H], 5.30 [m, 1H, C(1) H], 4.60 [m, 1H, C(2′) H], 4.21 [m, 1H, C(4′) H], 4.10 [d, 1H, J=5.2 Hz, C(6) H], 1.71 [s, 3H, C(3) CH₃], 1.43 [dd, 2H, J=7.6, 3.2 Hz, C(3″) H₂], 1.04 [s, 3H, C(2″) CH₃], 1.02 [s, 3H, C(2″) CH₃], 0.75 [t, 3H, J=7.5 Hz, C(4″) H₃], 0.74 [d, 3H, J=7.3 Hz, C(7) CH₃]. 100 MHz ¹³C NMR: δ (ppm) 178.03 [C(1″)], 171.76 [C(6′)], 141.87 [C(3)], 135.95 [C(4a)], 124.82 [C(4)], 117.88 [C(5)], 84.31 [C(6)], 77.24 [C(2′)], 68.27 [C(1)], 62.99 [C(4′)], 43.54 [C(2″)], 40.42 [C(8a)], 39.15 [C(5′)], 36.76 [C(2)], 36.20 [C(3′)], 33.80 [C(3″)], 33.64 [C(10)], 32.03 [C(8)], 30.86 [C(7)], 25.34 [C(9)], 25.28 [(C(2″-CH3)], 24.66 [(C(2″-CH₃)], 23.43 [C(3-CH₃)], 10.45 [C(7-CH₃)], 9.66 [C(4″)]. The proton of the hydroperoxide group at 9.64 ppm was confirmed by a D₂O exchange experiment and reduction to the corresponding 6-alcohol. DEPT 90, 135, HETCOR and COSY experiments supported the assignment of the structure above.

3(R)-hydroperoxysimvastatin: 400 MHz ¹H NMR (CD₃CN): δ (ppm) 8.68 [s, 1H, C(3) OOH], 6.05 [d, 1H, J=9.8 Hz, C(5) H], 5.98 [dd, 1H, J=9.6, 5.9 Hz, C(6) H], 5.53 [m, 1H, C(4) H], 5.11 [m, 1H, C(1) H], 4.55 [m, 1H, C(2′) H], 4.22 [m, 1H, C(4′)H], 2.54 [m, 1H, C(2) H_{α}], 2.33 [m, 1H, C(8a) H], 1.61 [m, 1H, C(2) H_{β}], 1.30 [s, 3H, C(3) CH₃], 1.11 [s, 3H, C(2″) CH₃], 0.90 [d, 3H, J=7.0 Hz, C(7) CH₃], 0.84 [t, 3H, J=7.4 Hz, C(4″) H₃]. 100 MHz ¹³C NMR: δ (ppm) 178.76 [C(1″)], 171.74 [C(6′)], 138.24 [C(6)], 137.81 [C(4a)], 129.06 [C(5)], 126.31 [C(4)], 78.69 [C(3)], 77.58 [C(2′)], 68.05 [C(1)], 63.49 [C(4′)], 44.01 [C(2″)], 39.66 [C(2)], 38.83 [C(8a)], 37.87 [C(7)], 36.91 [C(5′)], 36.31 [C(3′)], 34.09 [C(3″)], 33.92 [C(10)], 32.05 [C(8)], 26.99 [C(3-CH3)], 25.42 [(C(2″-CH₃)], 25.26 [(C(2″-CH₃)], 25.24 [C(9)], 14.07 [C(7-CH3)], 10.13 [C(4″)]. The signal for 3-CH₃, which appeared as a doublet at 1.08 ppm (1H) in the ¹H NMR spectrum of simvastatin was absent in the spectrum of this isolate. Instead, a new singlet was observed at 1.30 ppm. This singlet had a correlated ¹³C NMR signal at 27.0 ppm as revealed by HETCOR. ¹³C and DEPT 135 experiments showed that carbon 3 is a quaternary carbon. The appearance of a peak in the ¹³C NMR spectrum at 78.7 ppm implied addition of oxygen to carbon 3. NOESY was used to determine the stereochemistry at the C(3) position. Cross peaks were observed between 2.33 [C(8a) H] and 5.11 [C(1) H], 2.33 [C(8a) H] and 1.61 [C(2) H_{β}], 1.61 [C(2) H_{β}] and 5.11 [C(1) H], 5.11 [C(1) H] and 2.54 [C(2) H_{α}], 2.54 [C(2) H_{α}] 2.54 [C(2)H_{α}] and 1.61 [C(2) H_{β}], 1.61 [C(2) H_{β}] and 1.30 [C(3) CH₃], 1.30 [C(3) CH₃] and 1.61 [C(2) H_{β}]. The observed cross peaks support and are in complete agreement with the structural assignment.

3(S)-hydroperoxysimvastatin: 400 MHz ¹H NMR (CD₃CN): δ (ppm) 8.90 [s, 1H, C(3) OOH], 6.02 [d, 1H, J=9.5 Hz, C(5) H], 5.93 [dd, 1H, J=9.8, 6.0 Hz, C(6)H], 5.41 [b, 1H, C(4) H], 5.39 [m, 1H, C(1) H], 4.52 [m, 1H, C(2′) H], 4.20 [m, 1H, C(4′)H], 2.39 [m, 1H, C(8a) H], 2.29 [m, 1H, C(2) H_{β}], 2.08 [m, 1H, C(2) H_{α}], 1.23 [s, 3H, C(3) CH₃], 1.1054 [s, 3H, C(2″) CH₃], 1.1036 [s, 3H, C(2″) CH₃], 0.87 [d, 3H, J=7.0 Hz, C(7) CH₃], 0.82 [t, 3H, J=7.4 Hz, C(4″) H₃]. 100 MHz ¹³C NMR: δ (ppm) 178.11 [C(1″)], 171.30 [C(6′)], 137.58 [C(4a)], 137.32 [C(6)], 128.40 [C(5)], 127.57 [C(4)], 80.81 [C(3)], 77.22 [C(2′)], 70.55 [C(1)], 63.11 [C(4′)], 43.79 [C(2″)], 39.28 [C(2)], 38.08 [C(8a)], 37.34 [C(7)], 36.95 [C(5′)], 36.52 [C(3′)], 33.89 [C(3″)], 33.44 [C(10)], 31.54 [C(8)], 26.75 [C(3-CH₃)], 25.15 [(C(2″-CH3)], 24.75 [C(9)], 13.78 [C(7-CH₃)], 9.77 [C(4″)]. The 1D and 2D NMR data obtained for this isolate is essentially identical to that of the 3(R)-OOH suggesting that these two compounds are epimers. To determine the stereochemistry at carbon C(3), the approach taken was the same as for the characterization of the 3(R)-OOH degradate. All the expected correlations between protons 8a, 1, 2, 4 and 3-CH₃ were observed. The most significant correlations to determine the stereochemistry at (C3) were between the following pairs: 2.39 [C(8a) H] and 2.29 [C(2) H_{β}], and between 1.23 [C(3) CH₃] and 2.08 [C(2) H_{α}]. No correlation between 2.29 [C(2) H_{β}] and 1.23 [C(3) CH₃] suggested that [C(8a) H] and 2.29 [C(2) H_{β}] were on the same side of the ring. Finally, 1.23 [C(3)CH₃] showed correlation with 2.08 [C(2)H_{α}], indicating that the methyl group in this degradate was in the α-configuration.

Comparison of LC/MS and LC/UV Spectra of Simvastatin/BHA Reaction Product with the Experimental Simvastatin Formulation

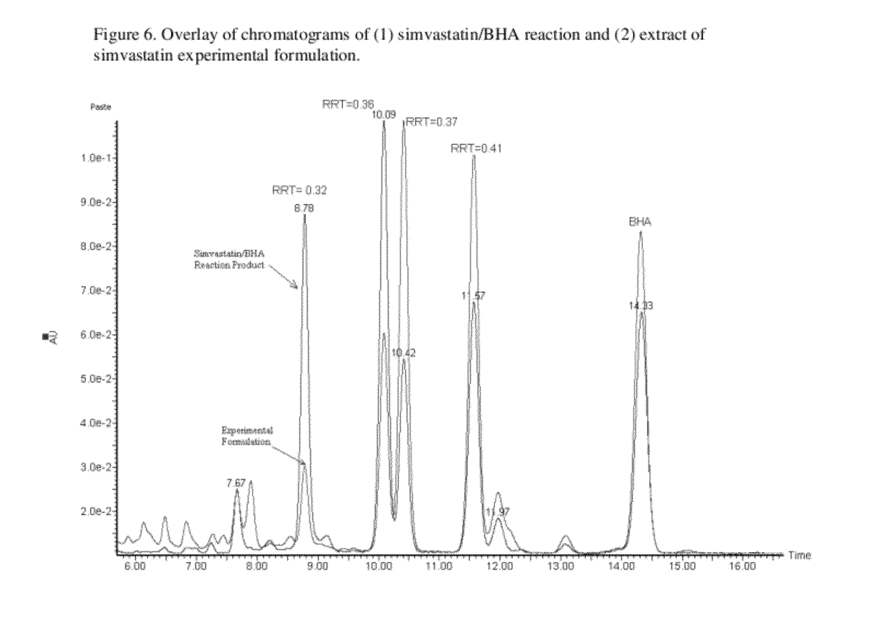

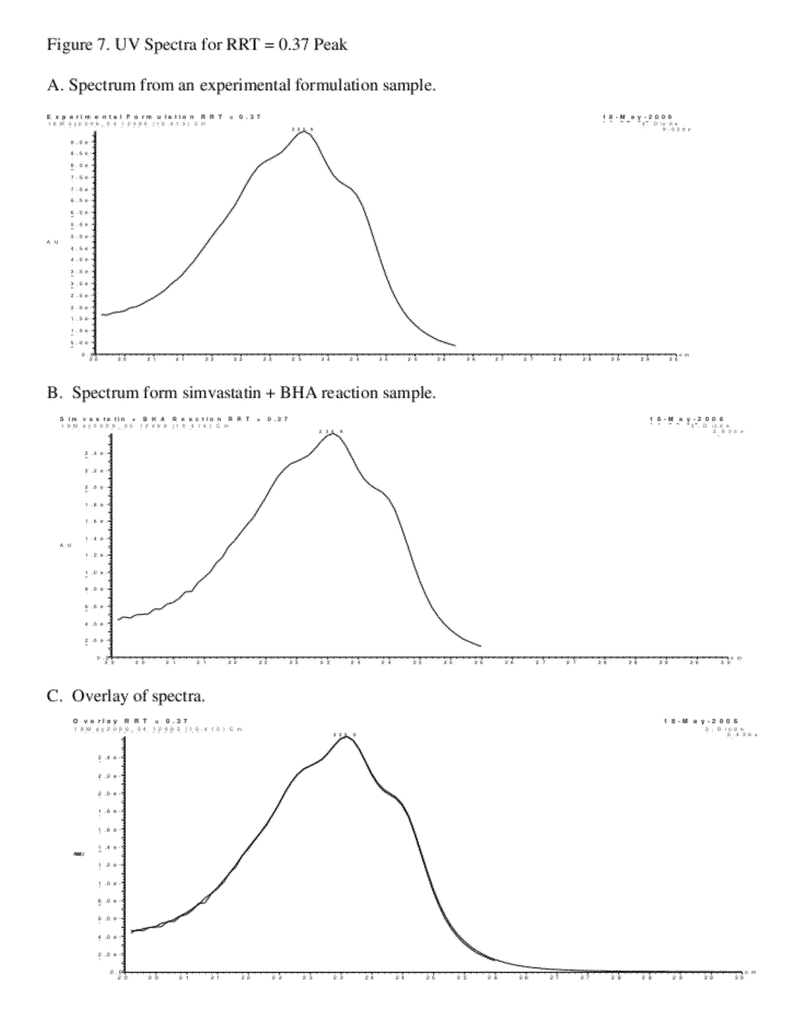

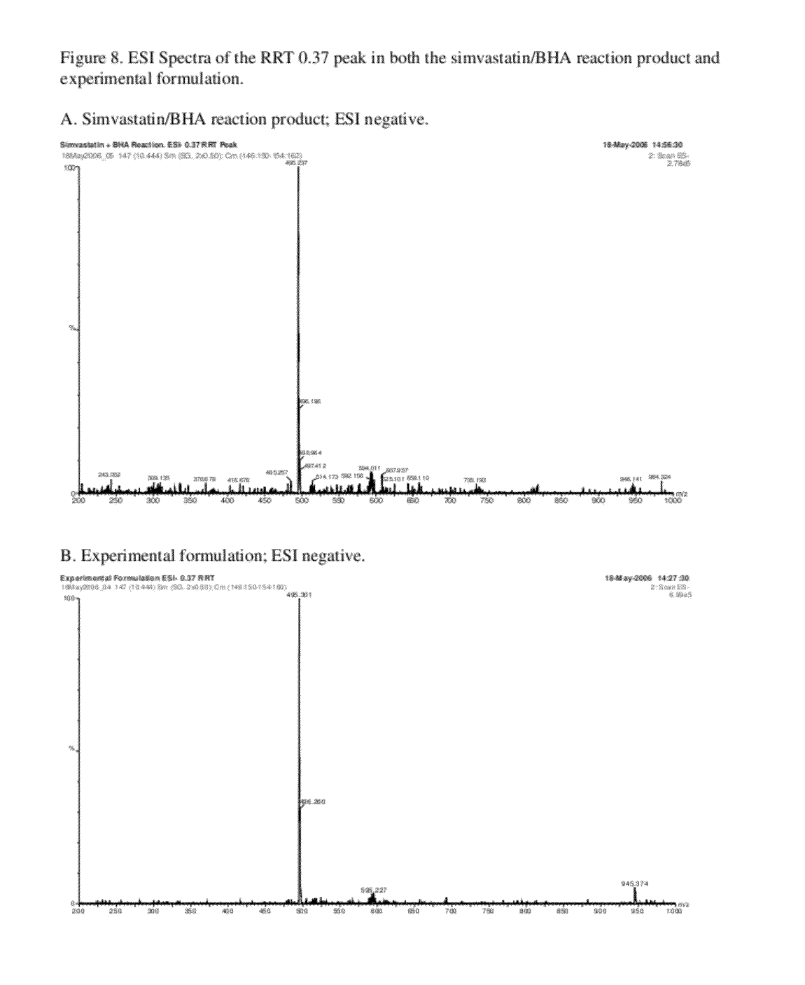

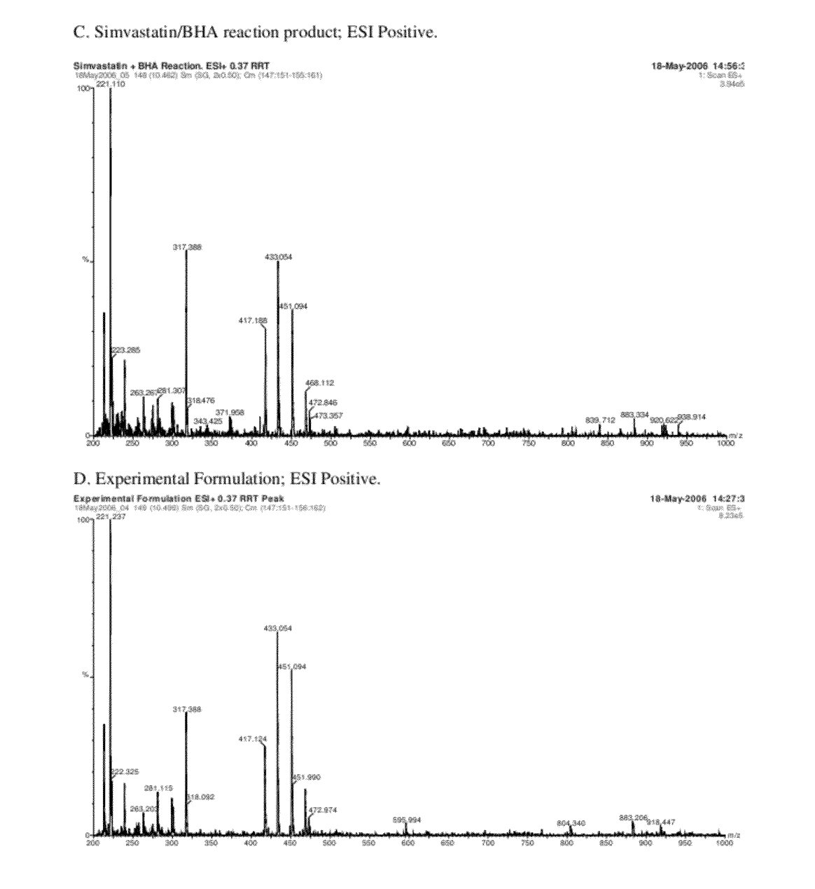

The ultraviolet and mass spectral characteristics of the chromatographic peaks from a simvastatin/BHA reaction sample were compared to those from the experimental simvastatin formulation sample. FIG. 6 displays superimposed chromatograms of the two samples with detection at 238. The chromatograms demonstrate the agreement in retention times for the four analytes. A comparison of the ultraviolet spectrum for 3(S)-hydroperoxysimvastatin (RRT=0.37) from the two samples is shown in FIG. 7. The figure contains (1) the spectrum from the experimental drug formulation sample maintained at 25° C. for nine months, (2) the simvastatin/BHA reaction sample and (3) the superimposition of the two spectra demonstrating agreement in spectral characteristics. Comparisons of spectral characteristics for the other three chromatographic peaks (not shown) from reaction product isolates and experimental simvastatin formulation samples were also in agreement. FIG. 8 shows the negative and positive ESI spectra of the RRT 0.37 peak for the two samples. The spectra for the products from the simvastatin/BHA reaction sample match those from the experimental drug formulation for both ionization modes. For each peak, the superimposed spectra demonstrate an excellent match between the experimental drug formulation and the simvastatin/BHA reaction product. The purified isolates were also spiked individually into acetonitrile extracts of the experimental drug formulation to establish consistency in retention times and further confirm the identities of the impurities in the experimental drug formulation. These results establish that the products produced from reaction of simvastatin and BHA were the same reaction products produced in the experimental drug formulation sample.

Reactivity of Statins with Antioxidants

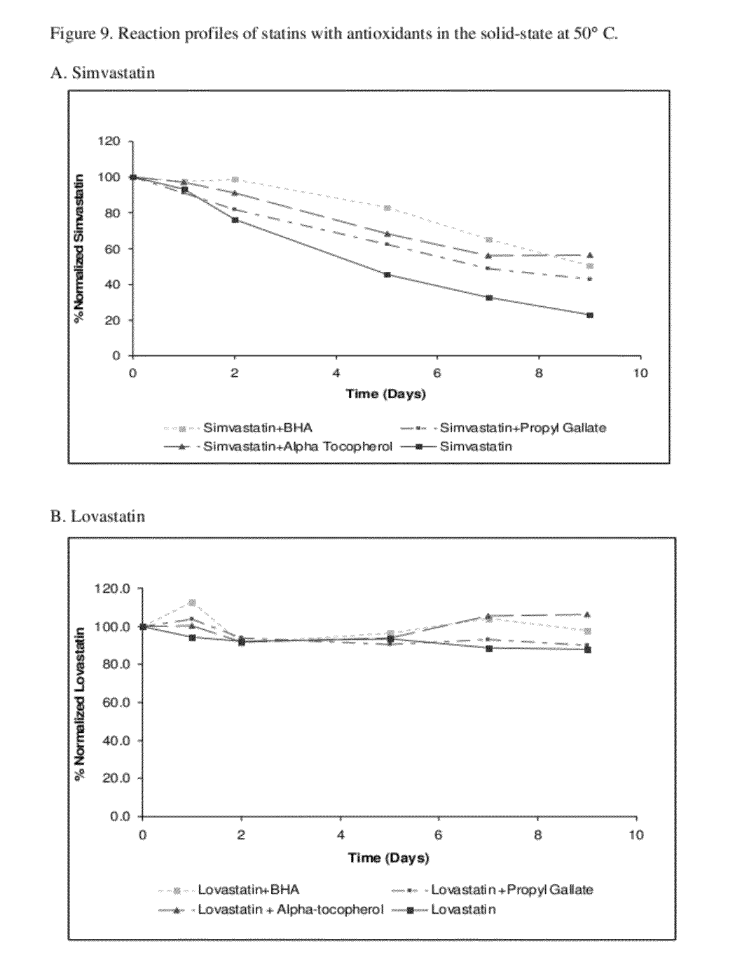

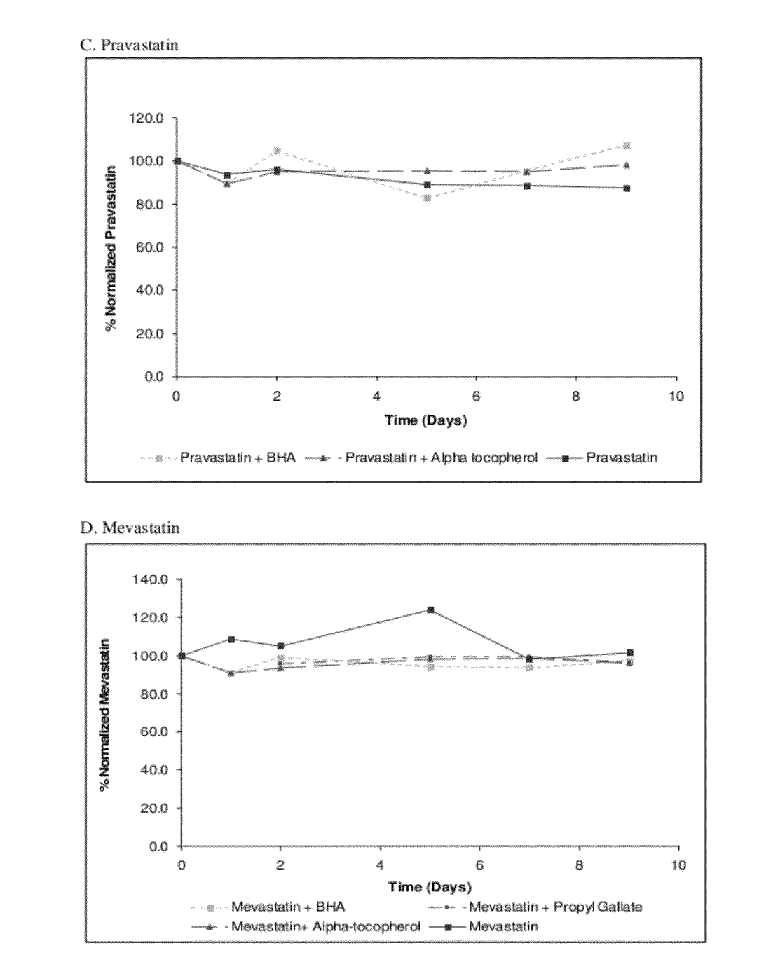

Reactions of the four statins showed significantly different reactivities with the antioxidants. FIG. 9 shows the results for the solid-state reactions performed at 50° C. over a period of 9 days. The concentration of simvastatin decreased significantly when incubated alone and with propyl gallatc, α-tocopherol and BHA. The concentration of lovastatin decreased about 12% when incubated alone and together with propyl gallate, but remained essentially unchanged when reacted with α-tocopherol and BHA. The concentration of pravastatin decreased approximately 18% when incubated alone. When reacted with BHA and α-tocopherol, the concentration of pravastatin remained essentially unchanged. The reaction between pravastatin and propyl gallate was not conducted because propyl gallate and pravastatin co-eluted during the HPLC analysis. Conversely, mevastatin showed no decrease in concentration when incubated alone or when reacted with antioxidants.

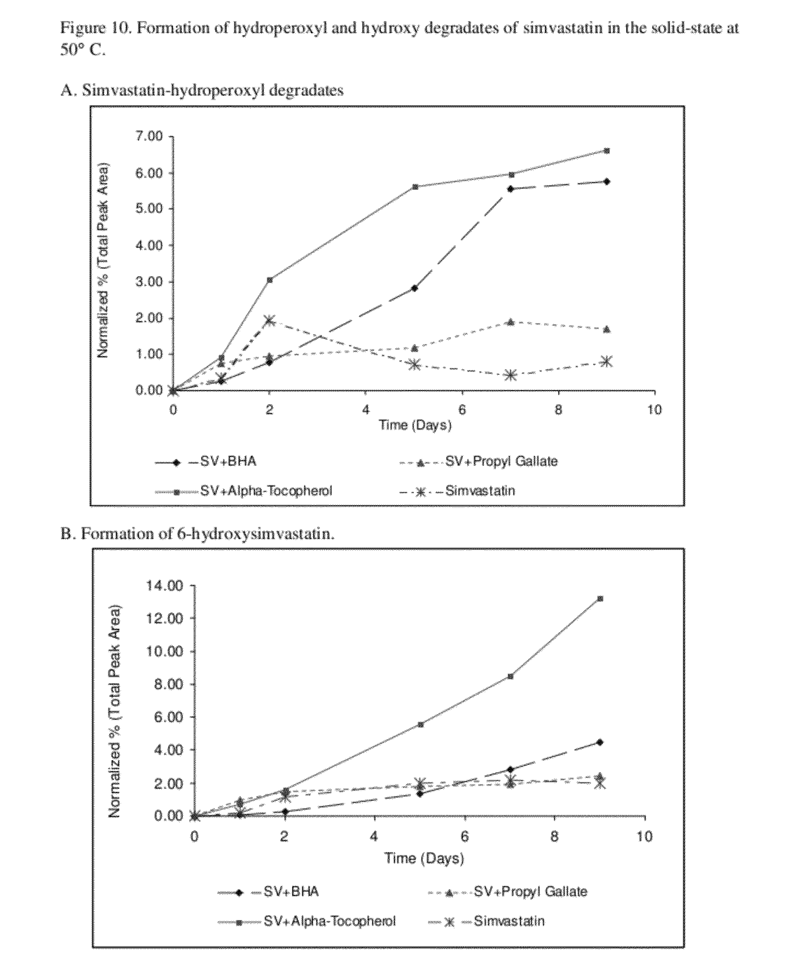

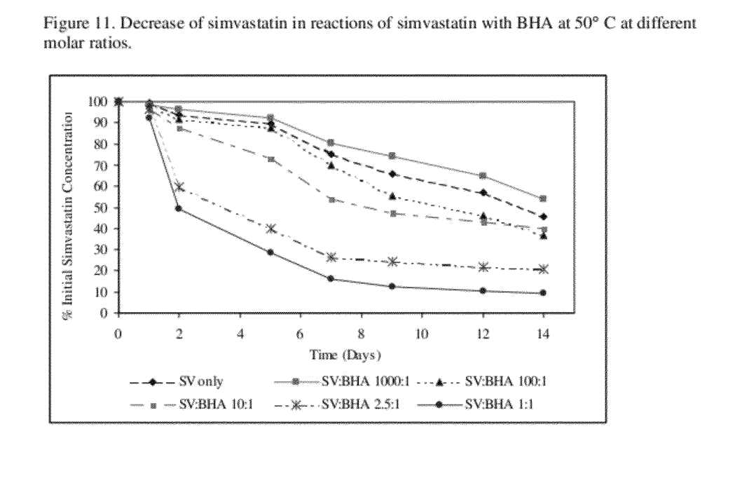

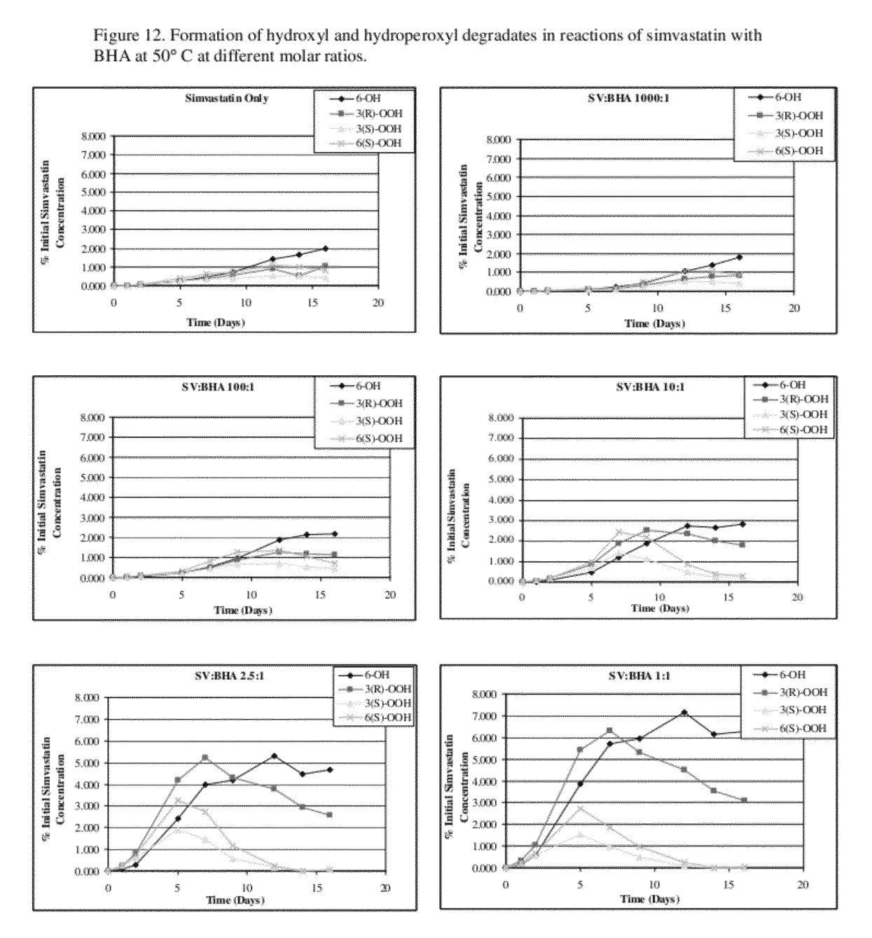

Formation of total hydroperoxyl degradates in the solid phase at 50° C. versus time is plotted in FIG. 10A. The total hydroperoxyl degradates is the sum of the concentrations of the 3(R), 3(S) and 6(S) simvastatin hydroperoxides. Reaction of simvastatin with α-tocopherol produced the highest concentrations of hydroperoxides throughout the nine-day incubation. Reaction with BHA produced hydroperoxides more slowly initially however after nine days the total hydroperoxide concentration was slightly lower than hydroperoxides produced with α-tocopherol. Propyl gallate with simvastatin, and simvastatin alone produced similar concentrations of hydroperoxide products. The production of 6(S)-hydroxysimvastatin from the reactions are show in FIG. 10B. α-tocopherol produced the highest concentrations of 6-hydroxysimvastatin throughout the nine day duration. Reactions with BHA, propyl gallate and simvastatin alone produced lower concentrations of 6-hydroxysimvastatin. The simvastatin:BHA ratio affected both the rate of simvastatin degradation (FIG. 11) and the formation of hydroxyl and hydroperoxyl degradates (FIG. 12). Reactions of lovastatin, mevastatin, and pravastatin with antioxidants produced minimal quantities of hydroxy and hydroperoxide products as determined by LC/MS analyses. Isolation and characterization of these products were not attempted due to the low concentrations.

### Results

Simvastatin was significantly more reactive than lovastatin, sodium pravastatin or mevastatin. Simvastatin decomposed to a greater extent than the other statins and produced more hydroxyl and hydroperoxyl degradates when reacted with α-tocopherol, BHA and propyl gallate. The same hydroxyl and hydroperoxyl degradates of simvastatin were produced from the different antioxidants (α-tocopherol, BHA, and propyl gallate). In the absence of any antioxidant, degradation of simvastatin was observed, yet no significant quantities of degradates were produced which resulted in a poor mass balance. Only low concentrations hydroxyl and hydroperoxyl degradates were detected. Minimal degradation of lovastatin, sodium pravastatin and mevastatin was observed when reacted with α-tocopherol, BHA and propyl gallate. LC/MS analyses of the reaction matrix indicated the presence of very low concentrations of hydroxyl and hydroperoxyl products in these reactions. Isolation and characterization of these compounds was not attempted due to the minimal concentrations of these compounds.

When comparing molecular structures, simvastatin is most similar to lovastatin in that it differs by one methyl group at the 2-carbon of the butanoic ester group yet the reactivity between the two compounds was significantly different. In contrast, a study of the oxidative susceptibility of statins in aqueous solution using a radical initiator showed that lovastatin and simvastatin had nearly identical rates of oxidation. Since crystal morphology can affect reactivity with oxygen¹, simvastatin was reacted with BHA in acetonitrile solvent at 50° C. for comparison to the solid-state results. This solution reaction with BHA produced comparable quantities of hydroperoxyl products and 6-hydroxysimvastatin to the solid-state reactions indicating that the difference in reactivity observed was not related to the solid form of simvastatin in the solid-state reactions.

As shown in FIG. 12, formation of simvastatin hydroxy and hydroperoxy degradates increased as the concentration of BHA increased in the solid-state reactions. To rule out an impurity in BHA as the cause of this reactivity, BHA was purified by sublimation. Reactions with the sublimed BHA produced similar simvastatin degradation and product formation profiles when compared to reactions with non-sublimed BHA.

Autoxidation of the statins at the conjugated diene presumably follows the conventional mechanism involving the radical initiation, propagation and termination steps'.

Initiation: In.+R—H→In—H+R. (1)

Propagation: R.+O₂→ROO. (2)

ROO.+R—H→R.+ROOH (3)

Termination: ROO.→Products (4)

The phenolic antioxidants are considered chain breaking by the donation of H. to the peroxyl radical:

ROO.+ArOH→ROOH+ArO. (5)

The resonance stabilized phenoxyl radicals do not typically propagate the chain reaction but are eventually consumed by reaction with a second peroxyl radical. In the presence of α-tocopherol, BHA, or propyl gallate the greater yield of hydroperoxyl degradants of simvastatin may be attributed to reaction (5). Simvastatin peroxy radicals are presumably short-lived and formation of the potentially more stable hydroperoxides would account for accumulation of these products. Increased production of the hydroperoxide degradates at high BHA:simvastatin ratios may be attributed to a prooxidant effect by the antioxidants. Some antioxidants under specific conditions have been reported to behave as prooxidants thus increasing oxidation of the substrate. Antioxidants have been shown to act as prooxidants at high concentrations during the autoxidation of polyunsaturated fatty acids. α-tocopherol trapped out the kinetic peroxyl radicals thus altering the stereochemistry of the hydroperoxide products formed. Additionally, tocopherols may inhibit decomposition of lipid hydroperoxide. The prooxidant effect was attributed to hydrogen atom abstraction by α-tocopherol radicals and rationalized by Reactions 6 and 7:

ArO.+R—H→ArOH+ROO. (6)

ArO.+ROOH→ROO.+ArOH (7)

Reaction 6 generates another peroxyl radical to propagate the chain rationalizing the prooxidant ability of the antioxidant. In the presence of antioxidants, the termination step (Reaction 8) would be minimized due to the predominance of hydroperoxides. Formation of hydroxyl degradates may be attributed to the decomposition of hydroperoxyl degradates possible via homolysis or other mechanisms.

2ROO.→Products (8)

Reactions of simvastatin with BHA in solution and solid-state were performed under an oxygen atmosphere at 2000 psi in a Parr pressure vessel at 50° C. The yield of the hydroxyl and hydroperoxyl products were essentially identical to reactions performed at ambient conditions. The rate of Reaction (2) is generally accepted as being fast in solution with rate constants on the order of 10⁹ Msec⁻¹ at 300K. These results indicate that for these reactions this step was not rate-limiting.

Solid-state reactions in formulations is a challenge in pharmaceutical research and development. Oxidation and other reactions are common degradation pathways encountered in the solid-state. Antioxidants are commonly used to provide stability to a formulation however, as this work showed, careful selection of the antioxidant or combination of antioxidants is essential since the antioxidant-effect may be specific to the active drug. Additionally, minor changes in formulation components could potentially alter the reactivity of the drug or drug/antioxidant system. Prooxidant effects are difficult to predict and must be considered during formulation development. Evaluation of the reactivity of the drug and candidate antioxidant in the absence of excipients provides basic information related to potential reactivity problems.

We claim:

1. The use of butylated hydroxy anisole (BHA) to reduce oxidative degradation of simvastatin.

2. The use of propyl gallate to reduce oxidative degradation of simvastatin.

3. The use of α-tocopherol to reduce oxidative degradation of simvastatin.
